(19) United States
(12) Patent Application Publication
(10) Pub. No.: US 2008/0206215 A1
(43) Pub. Date: Aug. 28, 2008

(54) APPARATUS AND METHOD FOR TREATMENT OF MICROORGANISMS DURING PROPAGATION, CONDITIONING AND FERMENTATION

(76) Inventor: Allen Michael Ziegler, Littleton, CO (US)

Correspondence Address: MCANDREWS HELD & MALLOY, LTD, 500 WEST MADISON STREET, SUITE 3400, CHICAGO, IL, 60661, US

(21) Appl. No.: 11/626,172

(22) Filed: Jan. 23, 2007

Related U.S. Application Data
(60) Provisional application No. 60/775,615, filed on Feb. 22, 2006.

(51) Int. Cl. A61K 35/66; C12M 1/00; A61P 43/00

(52) U.S. Cl. 424/93.51; 435/289.1; 435/303.1

(57) ABSTRACT

A method of reducing undesirable microorganism concentration, promoting desirable microorganism propagation/conditioning, and increasing desirable microorganism efficiency in an aqueous fluid stream includes (a) introducing a quantity of fermentable carbohydrate or cellulose to an aqueous fluid stream, (b) introducing a quantity of desirable microorganism to the aqueous fluid stream, (c) generating ClO₂ gas, (d) dissolving the ClO₂ gas to form a ClO₂ solution, and (e) introducing an aqueous ClO₂ solution into the aqueous fluid stream. Another method includes (a) introducing a quantity of fermentable carbohydrate or cellulose to an aqueous fluid stream, (b) introducing a quantity of desirable microorganism to the aqueous fluid stream, and (c) introducing ClO₂ having an efficiency as ClO₂ of at least about 90% into the aqueous fluid stream. An apparatus for reducing bacteria concentration, promoting fungi propagation/conditioning, and increasing yeast efficiency comprises a ClO₂ generator fluidly connected to a batch tank, fluidly connected to a fungi vessel.

### CROSS-REFERENCE TO RELATED APPLICATIONS(S)

This application relates to and claims priority benefits from U.S. Provisional Patent Application Ser. No. 60/775,615, filed Feb. 22, 2006, entitled “Apparatus And Method For Treatment Of Yeast During Propagation, Conditioning And Fermentation”. The '615 provisional application is hereby incorporated by reference herein in its entirety.

### FIELD OF THE INVENTION

Generally, the technical field involves anaerobic and aerobic microbial propagation, conditioning and/or fermentation. Specifically, it is a method of reducing the concentration of undesirable microorganisms while simultaneously encouraging propagation and/or conditioning of desirable microorganisms and increasing the efficiency of desirable microorganisms during fermentation.

### BACKGROUND OF THE INVENTION

Microorganisms, such as yeast, fungi and bacteria, are used to produce a number of fermentation products, such as industrial grade ethanol, distilled spirits, beer, wine, pharmaceuticals and nutraceuticals (foodstuff that provides health benefits, such as fortified foods and dietary supplements). Yeast are also commonly utilized in the baking industry.

Yeast are the most commonly used microorganism in fermentation processes. Yeast are minute, often unicellular, fungi. They usually reproduce by budding or fission. One common type of yeast is Saccharomyces cerevisia, the species predominantly used in baking and fermentation. Non-Sacharomyces yeasts, also known as non-conventional yeasts, are also used to make a number of commercial products. Some examples of non-conventional yeasts include Kuyberomyces lactis, Yarrowia lipolytica, Hansenula polymorpha and Pichia pastoris.

However, other microorganisms can also be useful in making fermentation products. For example, cellulosic ethanol production, production of ethanol from cellulosic biomass, utilizes fungi and bacteria. Examples of these cellulolytic fungi include Trichoderma reesei and Trichoderma viride. One example of a bacteria used in cellulosic ethanol production is Clostridium Ijungdahlii.

Most of the yeast used in distilleries and fuel ethanol plants are purchased from manufacturers of specialty yeasts. The yeast are manufactured through a propagation process. Propagation involves growing a large quantity of yeast from a small lab culture of yeast. During propagation, the yeast are provided with the oxygen, nitrogen, sugars, proteins, lipids and ions that are necessary or desirable for optimal growth through aerobic respiration.

Once at the distillery, the yeast can undergo conditioning. The objective of both propagation and conditioning is to deliver a large volume of yeast to the fermentation tank with high viability, high budding and a low level of infection by other microorganisms. However, conditioning is unlike propagation in that it does not involve growing a large quantity from a small lab culture. During conditioning, conditions are provided to re-hydrate the yeast, bring them out of hibernation and allow for maximum anaerobic growth and reproduction.

Following propagation or conditioning, the yeast enter the fermentation process. The yeast are combined in an aqueous solution with fermentable sugars. The yeast consume the sugars, converting them into aliphatic alcohols, such as ethanol.

During these three processes the yeast can become contaminated with bacteria or other undesirable microorganisms. This can occur in one of the many vessels used in propagation, conditioning or fermentation. This includes propagation tanks, conditioning tanks, starter tanks, fermentations tanks, piping and heat exchangers between these units.

Bacterial or microbial contamination reduces the fermentation product yield in three main ways. First, the sugars that could be available for yeast to produce alcohol are consumed by the bacteria or other undesirable microorganisms and diverted from alcohol production. In addition to reducing yield, the end products of bacterial metabolism, such as lactic acid and acetic acid, inhibit yeast growth and yeast fermentation/respiration, which results in less efficient yeast production. Finally, the bacteria or other undesirable microorganisms compete with the yeast for nutrients other than sugar.

After the fermentation stream or vessel has become contaminated with bacteria or other undesirable microorganisms, those bacteria or other microorganisms can grow much more rapidly than the desired yeast. The bacteria or other microorganisms compete with the yeast for fermentable sugars and retard the desired bio-chemical reaction resulting in a lower product yield. Bacteria also produce unwanted chemical by-products, which can cause spoilage of entire fermentation batches. Removing these bacteria or other undesirable microorganisms allows the yeast to thrive, which results in higher efficiency.

As little as a one percent decrease in ethanol yield is highly significant to the fuel ethanol industry. In larger facilities, such a decrease in efficiency will reduce income from 1 million to 3 million dollars per year.

Some previous methods of reducing bacteria or other undesirable microorganisms during propagation, conditioning and fermentation take advantage of the higher temperature and pH tolerance of yeast over other microorganisms. This is done by applying heat to or lowering the pH of the yeast solution. However, these processes are not entirely effective in retarding bacterial growth. Furthermore, the desirable yeast microorganisms, while surviving, are stressed and not as vigorous or healthy. Thus, the yeasts do not perform as well.

The predominant trend in the ethanol industry is to reduce the pH of the mash to less than 4.5 at the start of fermentation. Lowering the pH of the mash reduces the population of some species of bacteria. However it is much less effective in reducing problematic bacteria, such as lactic-acid producing bacteria, and is generally not effective for wild yeast and molds. It also significantly reduces ethanol yield by stressing the yeast.

Another current method involves the addition of antibiotics to the yeast propagation, conditioning or fermentation batch to neutralize bacteria. This method has a number of problems. Antibiotics are expensive and can add greatly to the costs of large-scale production. Improved technology that refines and improves the efficiency of existing techniques would be of considerable value to the industry. Moreover, antibiotics are not effective against all strains of bacteria, such as antibiotic-resistant strains of bacteria. Overuse of antibiotics can lead to the creation of additional variants of antibiotic-resistant strains of bacteria.

Antibiotic residues and establishment of antibiotic-resistant strains is a global issue. These concerns may lead to future regulatory action against the use of antibiotics. One area of concern is dried distillers grain that is used for animal feed. European countries do not allow the byproducts of an ethanol plant to be sold as animal feed if antibiotics are used in the facility. Dried distiller grain sales account for up to 20% of an ethanol plant earnings. Antibiotic concentration in the byproduct can range from 1-3% by weight, thus negating this important source of income.

In addition, there are other issues to consider when using antibiotics. Calculating the correct dosage of antibiotic can be a daunting task. Even after dosages have been determined, mixtures of antibiotics should be constantly or at least frequently balanced and changed in order to avoid single uses that will lead to antibiotic-resistant strains. Sometimes the effective amount of antibiotic cannot be added to the fermentation mixture. For example, utilizing over 2 mg/L of Virginiamycin will suppress fermentation but over 25 mg/L is required to inhibit grown of Weisella confusa, an emerging problematic bacteria strain.

Another approach involves washing the yeast with phosphoric acid. This method does not effectively kill bacteria and other microorganisms. It can also stress the yeast, thereby lowering their efficiency.

Yet another method is to use heat or harsh chemicals and sterilize process equipment between batches. However this method is only effective when equipment is not in use. It is ineffective at killing bacteria and other microorganisms within the yeast mixture during production.

Chlorine dioxide (ClO₂) has many industrial and municipal uses. When produced and handled properly, ClO₂ is an effective and powerful biocide, disinfectant and oxidizer.

ClO₂ has been used as a disinfectant in the food and beverage industries, wastewater treatment, industrial water treatment, cleaning and disinfections of medical wastes, textile bleaching, odor control for the rendering industry, circuit board cleansing in the electronics industry, and uses in the oil and gas industry. It is an effective biocide at low concentrations and over a wide pH range. ClO₂ is desirable because when it reacts with an organism in water, it reduces to chlorite ion and then to chloride, which studies to date have shown does not pose a significant adverse risk to human health.

Previously, brewers added an aqueous 2-6% by weight sodium chlorite solution, otherwise known as stabilized chlorine dioxide, to their fermentation batches in an attempt to kill bacteria and other microorganisms. When sodium chlorite reacts in an acidic environment it can form ClO₂. The ClO₂ added using this method was not substantially pure, which made it difficult to ascertain the amount added or control that amount with precision. If the amount is not precisely maintained, the ClO₂ can kill the desired yeast or inhibit the glucoamylase enzyme that is present to prepare the fermentable sugars. If these undesirable consequences occur, the addition of ClO₂ will not result in more efficient production. This method is also not effective at a neutral or basic pH level.

Producing ClO₂ gas for treating yeast during the propagation, conditioning and/or fermentation process is desirable because there is greater assurance of ClO₂ purity when in the gas phase. ClO₂ is, however, unstable in the gas phase and will readily undergo decomposition into chlorine gas (Cl₂), oxygen gas (O₂), and heat. The high reactivity of ClO₂ generally requires that it be produced and used at the same location.

Accordingly, it would be desirable to provide a less costly and more effective method of reducing undesirable microorganisms during propagation, conditioning and/or fermentation than those currently used. It is also desirable that this method encourage propagation and/or conditioning of the desirable microorganisms and increase their efficiency in fermentation. It is also desirable to avoid the use of antibiotics during yeast and/or microbial propagation, conditioning and/or fermentation. It is also desirable to avoid inhibition of glucoamylase during microbial propagation, conditioning and/or fermentation.

### SUMMARY OF THE INVENTION

A method for reducing undesirable microorganism concentration, promoting yeast propagation, and increasing yeast efficiency in an aqueous fluid stream comprises (a) introducing a quantity of fermentable carbohydrate to an aqueous fluid stream, (b) introducing a quantity of yeast to the aqueous fluid stream, (c) generating ClO₂ gas, (d) dissolving the ClO₂ gas to form a ClO₂ solution, and (e) introducing an aqueous ClO₂ solution into the aqueous fluid stream. These steps can be performed sequentially or in a different order.

In the foregoing method, the “undesirable” microorganisms intended to be reduced are those that compete for nutrients with the desirable microorganisms, such as yeast and Trichoderma that promote in the fermentation processes involved here. In this regard, the aqueous ClO₂ solution employed in the present method does not appear to detrimentally affect the growth and viability of desirable, fermentation-promoting microorganisms, but does appear to eliminate or at least suppress the growth of undesirable microorganisms that interfere with the fermentation process. Moreover, the elimination or suppression of undesirable microorganisms appears to have a favorable effect on the growth and viability of desirable microorganisms, for the reasons set forth in the Background section.

The ClO₂ gas can be generated by reacting chlorine gas with water and then adding sodium chlorite. Alternatively the ClO₂ gas could be generated by reacting sodium hypochlorite with an acid and then adding sodium chlorite. The ClO₂ gas can also be generated by reacting sodium chlorite and hydrochloric acid. The ClO₂ gas can also be generated using electrochemical cells and sodium chlorate or sodium chlorite. Equipment-based generation could also be used to create ClO₂ gas using sodium chlorate and hydrogen peroxide.

In one embodiment, the ClO₂ solution has a concentration of less than about 15 mg/L. In another embodiment the ClO₂ solution has a concentration of between about 10 and about 75 mg/L. In one embodiment the ClO₂ solution has an efficiency as ClO₂ in the stream of at least about 90%. As used in this application “to have an efficiency as ClO₂ of at least about 90%” means that at least about 90% of the ClO₂ solution or ClO₂ gas is in the form of ClO₂.

Another method that reduces undesirable microorganism concentration, promotes yeast propagation, and increases yeast efficiency in an aqueous fluid stream comprises (a) introducing a quantity of fermentable carbohydrate to an aqueous fluid stream, (b) introducing a quantity of yeast to the aqueous fluid stream, and (c) introducing ClO₂ having an efficiency as ClO₂ of at least about 90% into the aqueous fluid stream. These steps can be performed sequentially or in a different order.

The ClO₂ having an efficiency as ClO₂ in the stream of at least about 90% can be produced by equipment or non-equipment based methods. Examples of non-equipment based methods of ClO₂ generation include dry mix chlorine dioxide packets that include both a chlorite precursor packet and an acid activator packet. Equipment-based methods include using electrochemical cells with sodium chlorate or sodium chlorite, and a sodium chlorate/hydrogen peroxide method.

In one embodiment, the ClO₂ solution is in the form of an aqueous solution having a concentration of less than about 15 mg/L. In another embodiment the ClO₂ solution is in the form of an aqueous solution having a concentration of between about 10 and about 75 mg/L. In another embodiment the ClO₂ is in a gaseous form.

An apparatus for reducing undesirable microorganisms, promoting fungi propagation, and increasing fungi efficiency comprises a ClO₂ generator, a batch tank and a vessel for containing an aqueous fungi solution. The ClO₂ generator comprises an inlet for introducing at least one chlorine-containing feed chemical and an outlet for exhausting a ClO₂ gas stream from the generator. The batch tank is fluidly connected to the ClO₂ generator outlet and receives the ClO₂ gas stream from the ClO₂ generator outlet. The batch tank comprises an inlet for introducing a second water stream and an outlet for exhausting an aqueous ClO₂ solution from the batch tank. The vessel is fluidly connected to the batch tank. In operation, introducing the ClO₂ solution from the batch tank to the vessel promotes propagation of fungi present in the vessel.

The batch tank preferably has an inlet for introducing a second water stream and an outlet for exhausting an aqueous ClO₂ solution. In one preferred embodiment, the batch tank is capable of exhausting an aqueous ClO₂ solution that has a concentration of less than about 5,000 mg/L. In one embodiment, the exhausted ClO₂ solution is dosed to have a concentration between about 10 and about 50 mg/L. In another embodiment, the exhausted ClO₂ solution is dosed to have a concentration of less than about 15 mg/L. In yet another embodiment, the exhausted ClO₂ solution is dosed to have a concentration of less than about 50 mg/L.

The fungi vessel can be a conditioning tank, heatable, capable of performing liquefaction or a fungi propagation vessel. The fungi vessel could also be a fermentation tank having an inlet for fungi, an inlet for water, an inlet for fermentation chemicals and an outlet for the fermentation product connecting to processing equipment.

A method for reducing undesirable microorganism concentration, promoting desirable microorganism propagation, and increasing desirable microorganism efficiency in an aqueous fluid stream comprises (a) introducing a quantity of cellulose to an aqueous fluid stream, (b) introducing a quantity of desirable microorganisms to the aqueous fluid stream, (c) generating ClO₂ gas, (d) dissolving the ClO₂ gas to form a ClO₂ solution, and (e) introducing an aqueous ClO₂ solution into the aqueous fluid stream. These steps can be performed sequentially or in a different order. In one embodiment the ClO₂ solution has an efficiency as ClO₂ in the stream of at least about 90%.

Another method that reduces undesirable microorganism concentration, promotes desirable microorganism propagation, and increases desirable microorganism efficiency in an aqueous fluid stream comprises (a) introducing a quantity of cellulose to an aqueous fluid stream, (b) introducing a quantity of desirable microorganisms to the aqueous fluid stream, and (c) introducing ClO₂ having an efficiency as ClO₂ of at least about 90% into the aqueous fluid stream. These steps can be performed sequentially or in a different order.

Another method of reducing bacteria concentration without the use of antibiotics in an aqueous fluid stream employed in a fermentation process comprises (a)introducing a quantity of desirable microorganisms to said stream; and (b) introducing ClO₂ having an efficiency as ClO₂ of at least about 90% into said stream.

### BRIEF DESCRIPTION OF THE DRAWINGS

FIG. 1 is a flow diagram of the process for production of a fermentation product. Examples of points at which ClO₂ can be added to inhibit growth of microorganisms and promote yeast propagation are indicated.

FIG. 2 is a graph of time (in hours) versus ethanol produced (in grams) for fermentation batches treated with various concentrations of ClO₂ during fermentation.

FIG. 3 is a graph of time (in hours) versus ethanol produced (in grams) for mash treated with various concentrations of ClO₂ prior to the fermentation process.

FIG. 4 is a bar graph of viability (% of yeast cells living out of the original number) over time (in hours) in the corn mash treated with 0, 10 and 50 ppm of ClO₂.

FIG. 5 is a bar graph showing the amount of bacteria present (in CFU/g) in fermenting mash treated with different antimicrobial agents (in ppm) at different times (in hours).

FIG. 6 is a graph of the level of glucose produced by glucoamylase activity in a 5% maltose solution treated with different concentrations of chlorite ion (in mg/L) versus time (in minutes).

FIG. 7 is a schematic of fermentation process equipment with an integrated ClO₂ system in accordance with one embodiment.

### DETAILED DESCRIPTION OF PREFERRED EMBODIMENT(S)

The current disclosure relates to a method for reducing the concentration of bacteria and other undesirable microorganisms while simultaneously encouraging propagation and/or conditioning of desirable microorganisms and increasing the efficiency of those desirable microorganisms in fermentation and an apparatus for carrying out this method.

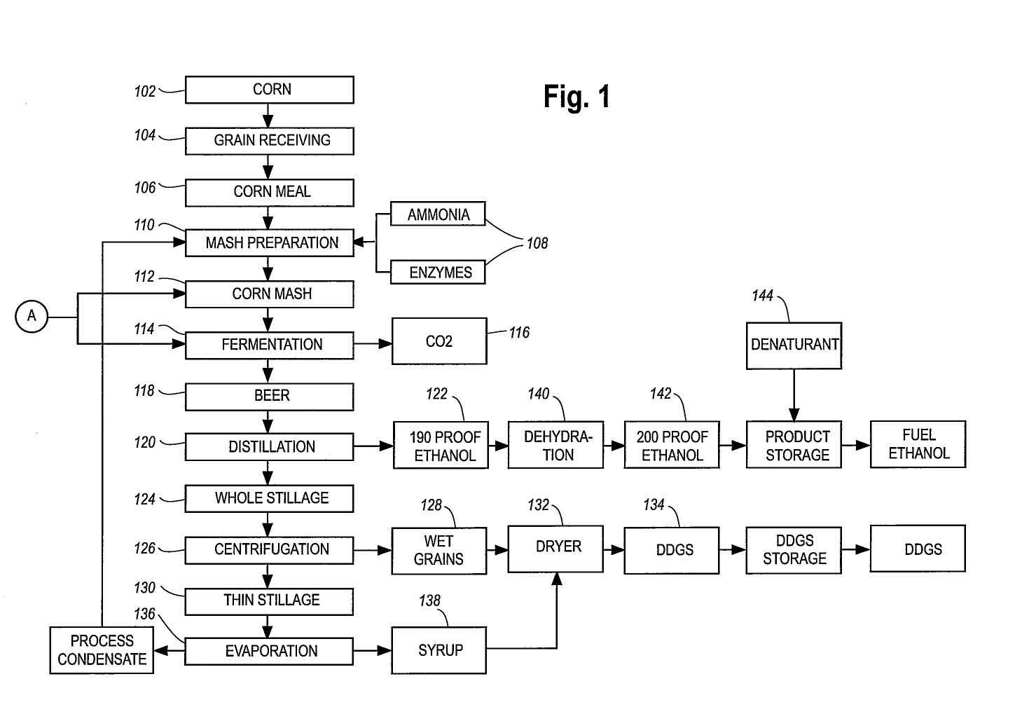

FIG. 1 illustrates the process for production of a fermentation product. The production of fuel ethanol by yeast fermentation is used as an example. However, this is merely one illustration and should not be understood as a limitation. Other fermentation products could include distilled spirits, beer, wine, pharmaceuticals, pharmaceutical intermediates, baking products, nutraceuticals (foodstuff that provides health benefits, such as fortified foods and dietary supplements), nutraceutical intermediates and enzymes. The current method could also be utilized to treat yeast used in the baking industry. Other fermenting microorganisms could also be substituted such as the fungi and bacteria typically used in cellulosic ethanol production, Trichoderma reesei, Trichoderma viride, and Clostridium Ijungdahlii.

The fermentation process begins with the preparation of a fermentable carbohydrate. In ethanol production, corn 102 is one possible fermentable carbohydrate. Other carbohydrates including cereal grains and cellulose-starch bearing materials, such as wheat or milo, could also be substituted. Cellulosic biomass such as straw and cornstalks could also be used. Cellulosic ethanol production has recently received attention because it uses readily available nonfood biomass to form a valuable fuel.

In corn-based ethanol production the corn is ground 104 into a fine powder called meal 106. The meal is then mixed with water and enzymes 108, such as alpha-amylase, and passed through a cooker in order to liquefy the starch 110. A product known as corn mash 112 results.

A secondary enzyme, such as glucoamylase 108, will also be added to the mash 112 to convert the liquefied starch into a fermentable sugar. The glucoamylase cleaves single molecules of glucose from the short chain starches, or dextrins. The glucose molecules can then be converted into ethanol during fermentation.

Yeast, small microorganisms capable of fermentation, will also be added to the corn mash 114. Yeast are fungi that reproduce by budding or fission. One common type of yeast is Saccharomyces cerevisia, the species predominantly used in baking and fermentation. Non-Sacharomyces yeasts, also known as non-conventional yeasts, are naturally occurring yeasts that exhibit properties that differ from conventional yeasts. Non-conventional yeasts are utilized to make a number of commercial products such as amino acids, chemicals, enzymes, food ingredients, proteins, organic acids, nutraceuticals, pharmaceuticals, cosmetics, polyols, sweeteners and vitamins. Some examples of non-conventional yeasts include Kuyberomyces lactis, Yarrowia lipolytica, Hansenula polymorpha and Pichia pastoris. The current methods and apparatus are applicable to intermediates and products of both Sacharomyces and non-conventional yeast.

Most of the yeast used in fuel ethanol plants and other fermentation processes are purchased from manufacturers of specialty yeast. The yeast are manufactured through a propagation process and usually come in one of three forms: yeast slurry, compressed yeast or active dry yeast. Propagation involves growing a large quantity of yeast from a small lab culture of yeast. During propagation the yeast are provided with the oxygen, nitrogen, sugars, proteins, lipids and ions that are necessary or desirable for optimal growth through aerobic respiration.

Once at the distillery, the yeast may undergo conditioning. The objectives of both propagation and conditioning are to deliver a large volume of yeast to the fermentation tank with high viability, high budding and a low level of infection by other microorganisms. However, conditioning is unlike propagation in that it does not involve growing a large quantity from a small lab culture. During conditioning, conditions are provided to re-hydrate the yeast, bring them out of hibernation and allow for maximum anaerobic growth and reproduction.

Following propagation or conditioning, the yeast enter the fermentation process. The glucoamylase enzyme and yeast are often added into the fermentation tank through separate lines as the mash is filling the fermentation tank. This process is known as simultaneous saccharification and fermentation or SSF. The yeast produce energy by converting the sugars, such as glucose molecules, in the corn mash into carbon dioxide 116 and ethanol.

The fermentation mash, now called “beer” 118 is distilled 120. This process removes the 190 proof ethanol, a type of alcohol, 122 from the solids, which are known as whole stillage 124. These solids are then centrifuged 126 to get wet distillers grains 128 and thin stillage 130. The distillers grains can be dried 132 and are highly valued livestock feed ingredients known as dried distillers grains (DDGS) 134. The thin stillage can be evaporated 136 to leave a syrup 138. After distillation, the alcohol is passed through a dehydration system 140 to remove remaining water. At this point the product is 200 proof ethanol 142. This ethanol is then denatured by adding a small amount of denaturant 144, such as gasoline, to make it unfit for human consumption.

The propagation, conditioning and fermentation processes can be carried out using batch and continuous methods. The batch process is used for small-scale production. Each batch is completed before a new one begins. The continuous fermentation method is used for large-scale production because it produces a continuous supply without restarting every time. The current method and apparatus are effective for both methods.

During the propagation, conditioning or fermentation process the mash or the fermentation mixture can become contaminated with other microorganisms, such as spoilage bacteria, wild yeast or killer yeast. These microorganisms compete with the yeast for fermentable sugars and retard the desired bio-chemical reaction resulting in a lower product yield. They can also produce unwanted chemical by-products, which can cause spoilage of entire fermentation batches. Wild yeast are a primary concern in the beverage industry because they can cause taste and odor problems with the final product. Killer yeast produce a toxin that is lethal to the desired alcohol producing yeast.

Producers of ethanol attempt to increase the amount of ethanol produced from one bushel of cereal grains, which weigh approximately 56 pounds (25.4 kilograms). Contamination by microorganisms lowers the efficiency of yeast making it difficult to attain or exceed the desired levels of 2.8-2.9 gallons per bushel (0.42-0.44 liters per kilogram). Reducing the concentration of microorganisms will encourage yeast propagation and/or conditioning and increase yeast efficiency making it possible to attain and exceed these desired levels.

Yeast can withstand and indeed thrive in a ClO₂ environment. However, bacteria, wild yeasts, killer yeasts and molds will succumb to the properties of ClO₂ allowing the producing, desirable yeast to thrive and achieve higher production

ClO₂ solution has many uses in disinfection, bleaching and chemical oxidation. ClO₂ can be added at various points in the propagation, conditioning and/or fermentation processes to kill unwanted microorganisms and promote growth and survival of the desirable microorganisms. This ClO₂ can be added as an aqueous solution or a gas. The ClO₂ can be added during propagation, conditioning and/or fermentation. The ClO₂ solution can be added to cook vessels, fermentation tanks, propagation tanks, conditioning tanks, starter tanks or during liquefaction. The ClO₂ solution can also be added to the interstage heat exchange system or heat exchangers. In one embodiment the ClO₂ has an efficiency as ClO₂ in the stream of at least about 90%. Adding ClO₂ having a known purity allows for addition of a controlled amount of ClO₂.

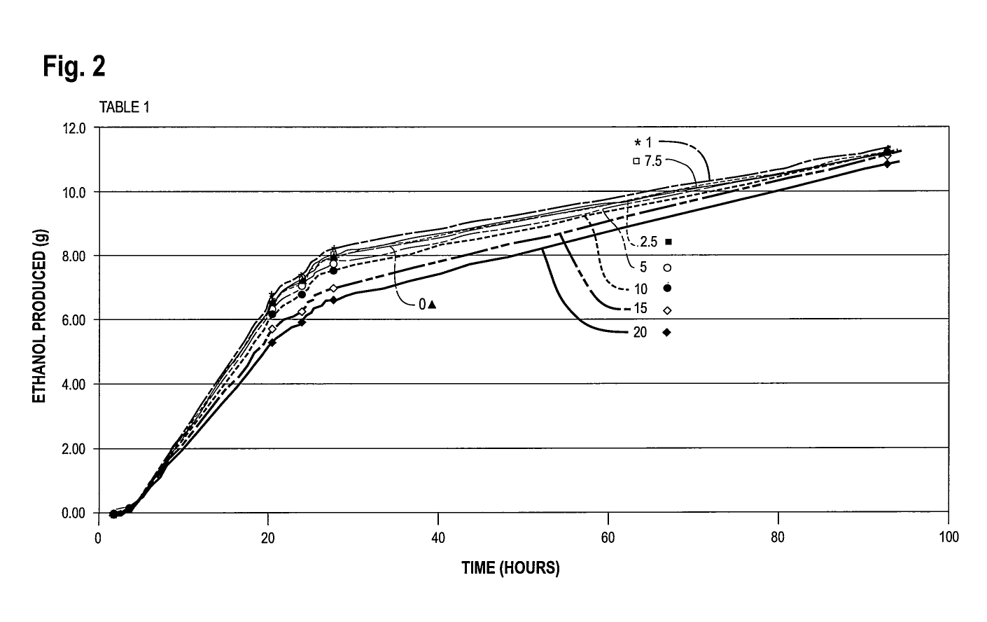

As mentioned above, ClO₂ can be added directly into the fermentation mixture. This can be done by adding the ClO₂ in conjunction with the yeast and glucoamylase, for example during the SSF stage. FIG. 2 is a graph of time (in hours) versus ethanol produced (in grams) for fermentation batches treated with various concentrations of ClO₂ during fermentation. This graph shows the relationship between addition of ClO₂ to a fermentation mixture and the amount of ethanol produced. Increases in ethanol production were noted with addition of ClO₂ during fermentation. Chlorine dioxide dosages of less than about 15 mg/L, preferably less than about 10 mg/L and most preferably less than about 7.5 mg/L applied directly to the fermentation mixture showed greater ethanol production than the control containing no ClO₂.

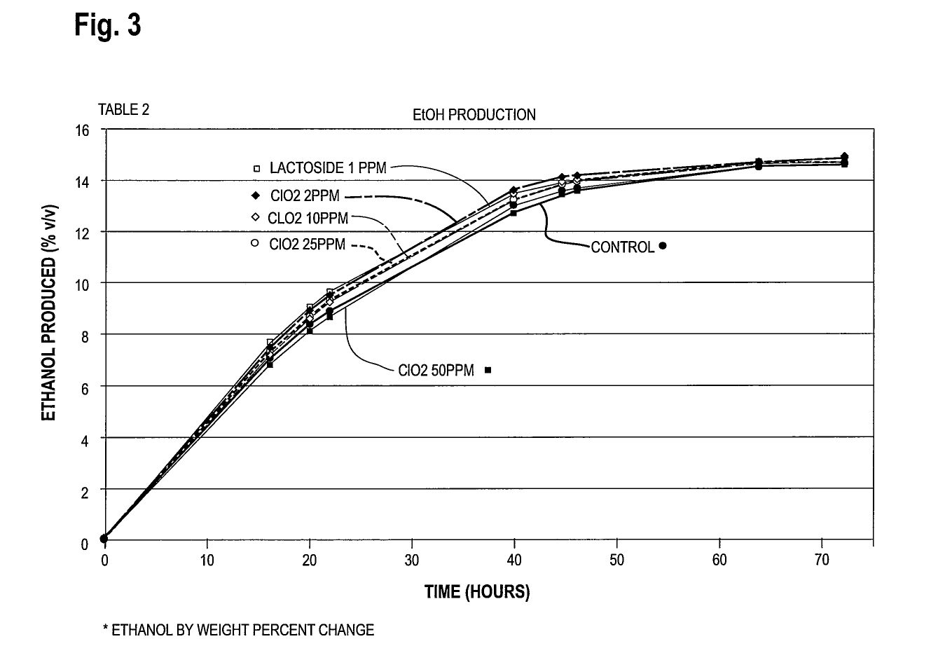

The ClO₂ can also be added to the mash prior to the fermentation process, for example before the SSF stage. FIG. 3 is a graph of time (in hours) versus ethanol produced (in grams) for mash treated with various concentrations of ClO₂ prior to the fermentation process. This graph shows the relationship between addition of ClO₂ to the corn mash prior to the fermentation process and the amount of ethanol produced. Increases in ethanol production were noted with addition of ClO₂ prior to fermentation. Chlorine dioxide dosages of between about 10 and about 75 mg/L, preferably between about 10 and about 50 mg/L and most preferable between about 20 and about 50 mg/L applied to the mash prior to fermentation showed greater ethanol production than the control containing no ClO₂.

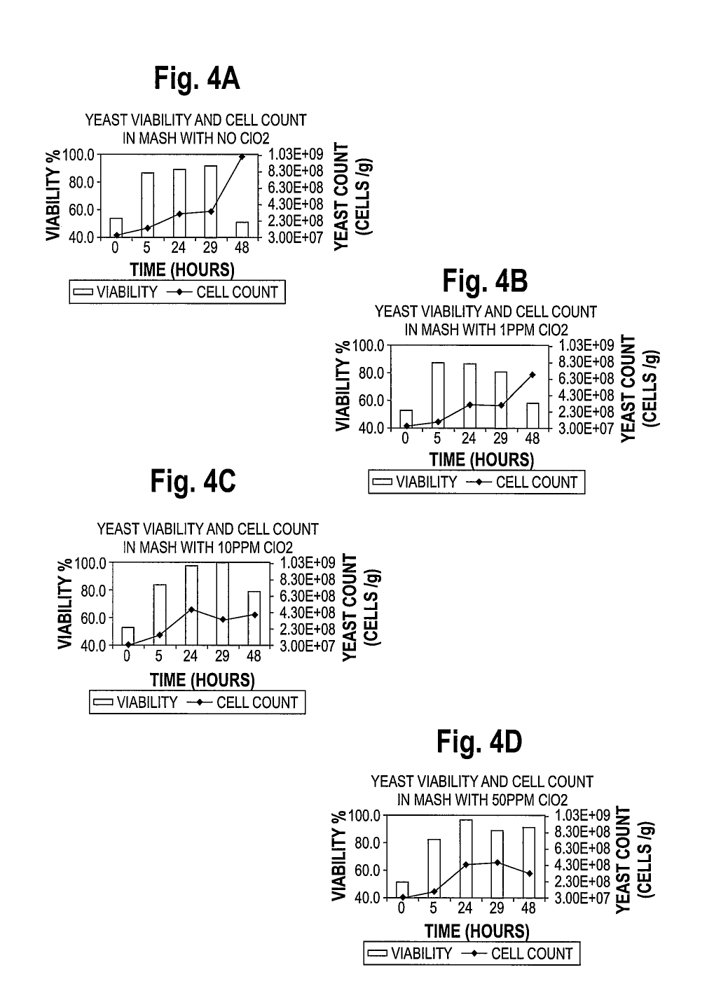

Chlorine dioxide can also be added during propagation and/or conditioning. For example ClO₂ can be added to the yeast slurry before SSF replacing the acid washing step. FIG. 4 is a bar graph of viability (% of yeast cells living out of the original number) over time (in hours) in the corn mash treated with 0, 10 and 500 ppm of ClO₂. This graph shows that yeast treated with ClO₂ during the propagation/conditioning phase exhibit up to 80% greater viability than untreated yeast. The yeast can tolerate a ClO₂ environment and remain viable at high concentrations of ClO₂. Competing bacteria, wild yeast, molds, etc. will succumb to the ClO₂ leaving only highly viable yeast for fermentation without the additional stress of traditional acid washing. Chlorine dioxide dosages of less than about 50 mg/L may be applied directly to the yeast during propagation.

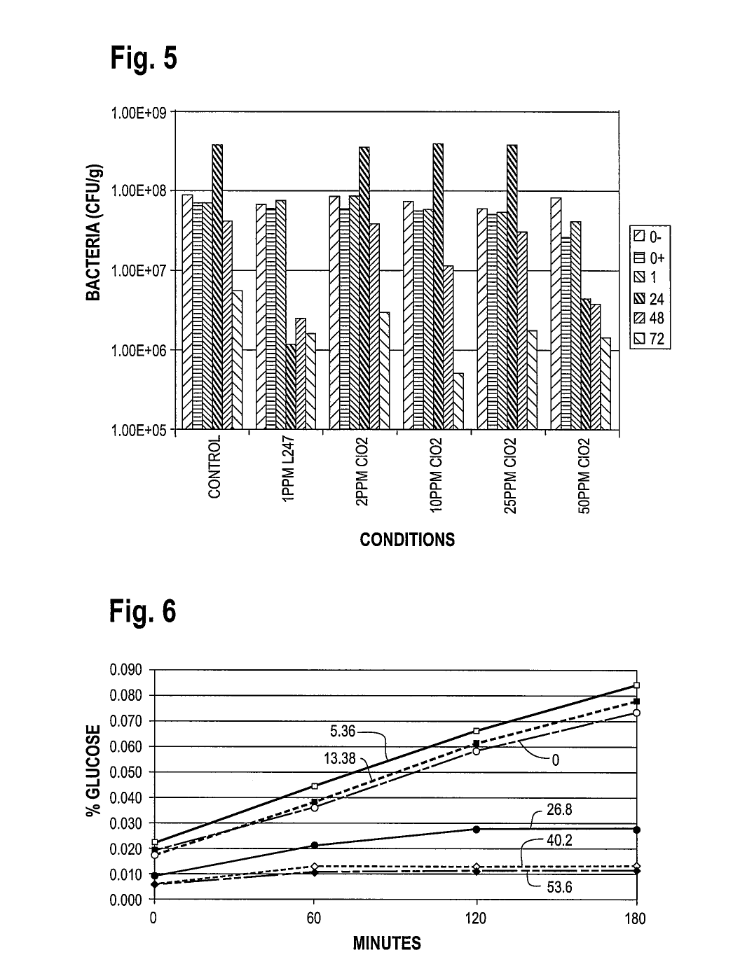

FIG. 5 is a bar graph showing the amount of bacteria present (in CFU/g) in fermenting mash treated with different levels of an antimicrobial agent (in ppm), either ClO₂ or antibiotic, at different times (in hours). This figure shows the effectiveness of ClO₂ as an antimicrobial agent. After 72 hours corn mash treated with ClO₂ exhibits greater microbial reduction than untreated mash. After 72 hours, the corn mash treated with greater than 10 ppm of ClO₂ also exhibits greater microbial reduction than the corn mash treated with antibiotic.

The ability of ClO₂ to attain or surpass the efficiency of antibiotics as an antimicrobial agent is a benefit of the current method. Numerous problems accompany the use of antibiotics as microbial agents in fermentation process. Antibiotics are expensive and are not effective against all strains of bacteria. Another area of concern is dried distillers grain that is used for animal feed. European countries do not allow the byproducts of an ethanol plant to be sold as animal feed if antibiotics are used in the facility. Dried distiller grain sales account for up to 20% of an ethanol plant earnings. Antibiotic concentration in the byproduct can range from 1-3% by weight, thus negating this important source of income.

In addition, there are other issues to consider when using antibiotics. Calculating the correct dosage of antibiotic can be a daunting task. Even after dosages have been determined, mixtures of antibiotics should be constantly or at least frequently balanced and changed in order to avoid single uses that will lead to antibiotic-resistant strains. The use of ClO₂ as an antimicrobial agent offers manufacturers a valuable option to antibiotics.

Another advantage of using ClO₂ as opposed to antibiotics deals with reduction byproducts. The ClO₂ reduces to form chlorite ion and then further reduces to form chloride ion and/or salt. The reduction from ClO₂ to chloride ion happens quickly and is indeterminate compared to the background residual already present. The chloride ion is a non-hazardous byproduct unlike those created by many antibiotics. Studies to date have shown that chloride ion does not pose a significant adverse risk to human health.

Since ClO₂ gas can decompose explosively, it is typically produced on-site. There are a number of methods of producing ClO₂ gas having a known purity, which are known to persons familiar with the technology involved here. One or more of these methods can be used. ClO₂ gas can be produced using electrochemical cells and a sodium chlorite or sodium chlorate solution. An equipment based sodium chlorate/hydrogen peroxide method also exists. Alternatively, non-equipment based binary, multiple precursor dry or liquid precursor technologies can be used. Examples of non-equipment based methods of ClO₂ generation include dry mix chlorine dioxide packets that include both a chlorite precursor packet and an acid activator packet. Other such processes include, but are not limited to, acidification of sodium chlorite, oxidation of chlorite by chlorine, oxidation of chlorite by persulfate, use of acetic anhydride on chlorite, use of sodium hypochlorite and sodium chlorite, use of dry chlorine/chlorite, reduction of chlorates by acidification in the presence of oxalic acid, reduction of chlorates by sulfur dioxide, and the ERCO R-2®, R-3®, R-5®, R-8®, R-10® and R-11® processes, from which ClO₂ is generated from NaClO₃ in the presence of NaCl and H₂SO₄ (R-2 and R-3 processes), from NaClO₃ in the presence of HCl (R-5 process), from NaClO₃ in the presence of H₂SO₄ and CH₃OH (R-8 and R-10 processes), and from NaClO₃ in the presence of H₂O₂ and H₂SO₄ (R-11 process).

Here, three methods will illustrate some possibilities. In the first method, chlorine reacts with water to form hypochlorous acid and hydrochloric acid. These acids then react with sodium chlorite to form chlorine dioxide, water and sodium chloride. In a second method, sodium hypochlorite is combined with hydrochloric or other acid to form hypochlorous acid. Sodium chlorite is then added to this reaction mixture to produce chlorine dioxide. The third method combines sodium chlorite and sufficient hydrochloric acid. In one embodiment the ClO₂ gas produced is between 0.0005 and 5.0% by weight in air.

The ClO₂ gas is dissolved in a solvent in order to create a ClO₂ solution. ClO₂ gas is readily soluble in water. In one embodiment the water and ClO₂ gas are combined in quantities that create a solution for application directly to the fermentation mixture, with a concentration of less than about 15 mg/L, preferably less than about 10 mg/L, and most preferably less than about 7.5 mg/L. In another embodiment the water and ClO₂ gas are combined in quantities that create a solution for application to the corn mash prior to fermentation, with a concentration of between about 10 and about 75 mg/L, preferably between about 10 and about 50 mg/L, and most preferable between about 20 and about 50 mg/L. In yet another embodiment the water and ClO₂ gas are combined in quantities that create a solution for application to the yeast during propagation with a concentration of less than about 50 mg/L. In the solution of one embodiment the ClO₂ solution has an efficiency as ClO₂ in the stream of at least about 90%.

Pure or substantially pure ClO₂ is desirable because it allows the user to precisely maintain the amount of ClO₂ added to the yeast. (The single term “pure” will be used hereinafter to mean either pure or substantially pure.) If too little ClO₂ is added the dosage will not be effective in killing undesirable microorganisms. If too much ClO₂ is added it can kill the desired yeast. If either of these situations occurs, the addition of ClO₂ will not result in more efficient ethanol production. Addition of pure ClO₂ allows the user to carefully monitor and adjust the amount of ClO₂ added to the yeast. This enables the user to add adequate ClO₂ to, assure microbial efficacy without killing the yeast.

Pure ClO₂ is also desirable for another reason. Glucoamylase enzyme is important in ethanol production to convert short chain starches (or dextrins) into fermentable glucose molecules. ClO₂ does not exhibit a significant reaction with glucoamylase. However, ClO₂ can reduce to form chlorite ion. FIG. 6 is a graph of the level of glucose (in % of maltose converted) produced by glucoamylase activity in a 5% maltose solution treated with different concentrations of chlorite ion (in mg/L) versus time (in minutes). FIG. 5 shows that the chlorite ion can inhibit the glucoamylase enzyme at approximately 14 mg/L and above. Inhibition of glucoamylase enzyme can lower ethanol production. A chlorite ion concentration of 14 mg/L can be produced by a ClO₂ dosage rate of about 50 to 60 mg/L. Addition of pure ClO₂ allows the user to add dosage rates below the level where glucoamylase inhibition can occur.

The ClO₂ solution is introduced at some point during the production of ethanol. The ClO₂ solution can be added during propagation, conditioning and/or fermentation. The ClO₂ solution can also be added directly to the corn mash. The ClO₂ solution can be added to cook vessels, fermentation tanks, propagation tanks, conditioning tanks, starter tanks or during liquefaction. The ClO₂ solution can also be added to the piping between these units or heat exchangers.

ClO₂ could also be used in the production of cellulosic ethanol. Cellulosic ethanol is a type of ethanol that is produced from cellulose, as opposed to the sugars and starches used in producing carbohydrate based ethanol. Cellulose is present in non-traditional biomass sources such as switch grass, corn stover and forestry. This type of ethanol production is particularly attractive because of the large availability of cellulose sources. Cellulosic ethanol, by the very nature of the raw material, introduces higher levels of contaminants and competing microorganism into the fermentation process. ClO₂ could be particularly helpful in cellulosic ethanol production as an antimicrobial agent.

There are two primary processes of producing alcohol from cellulose. One process is a hydrolysis process that utilizes a fungi such as Trichoderma reesei and Trichoderma viride. The other is a gasification process using a bacteria such as Clostridium Ijungdahlii. ClO₂ could be utilized in either process.

In the hydrolysis process the cellulose chains are broken down into five carbon and six carbon sugars before the fermentation process. This is either done chemically and enzymatically.

In the chemical hydrolysis method the cellulose can be treated with dilute acid at high temperature and pressure or concentrated acid at lower temperature and atmospheric pressure. In the chemical hydrolysis process the cellulose reacts with the acid and water to form individual sugar molecules. These sugar molecules are then neutralized and yeast fermentation is used to produce ethanol. ClO₂ could be used during the yeast fermentation portion of this method as outlined above.

Enzymatic hydrolysis can be carried out using two methods. The first is known as direct microbial conversion (DMC). This method uses a single microorganism to convert the cellulosic biomass to ethanol. The ethanol and required enzymes are produced by the same microorganism. ClO₂ could be used during the propagation/conditioning or fermentation steps with this specialized organism.

The second method is known as the enzymatic hydrolysis method. In this method cellulose chains are broken down using cellulase enzymes. These enzymes are typically present in the stomachs of ruminants, such as cows and sheep, to break down the cellulose that they eat. In this process the cellulose is made via fermentation by cellulolytic fungi such as Trichoderma reesei and Trichoderma viride.

The enzymatic method is typically carried out in four or five stages. The cellulose is pretreated to make the raw material, such as wood or straw, more amenable to hydrolysis. Next the cellulase enzymes are used to break the cellulose molecules into fermentable sugars. Following hydrolysis, the sugars are separated from residual materials and added to the yeast. The hydrolyzate sugars are fermented to ethanol using yeast. Finally, the ethanol is recovered by distillation. Alternatively, the hydrolysis and fermentation can be carried out together by using special bacteria or fungi that accomplish both processes. When both steps are carried out together the process is called sequential hydrolysis and fermentation (SHF).

ClO₂ is compatible with various Trichoderma fungi strains and can be introduced for microbiological efficacy at various points in the enzymatic method of hydrolysis. ClO₂ could be used in the production, manufacture and fermentation of cellulase enzymes made by Trichoderma and other fungi strains. The ClO₂ can be added in the cellulosic simultaneous saccharification and fermentation phase (SSF). The ClO₂ can be introduced in the sequential hydrolysis and fermentation (SHF) phase. It could also be introduced at a point before, during or after the fermentation by cellulolytic fungi that create the cellulase enzymes. Alternatively the ClO₂ could be added during the yeast fermentation phase, as discussed above.

The gasification process does not break the cellulose chain into sugar molecules. First, the carbon in the cellulose is converted to carbon monoxide, carbon dioxide and hydrogen in a partial combustion reaction. Then, the carbon monoxide, carbon dioxide and hydrogen are fed into a special fermenter that uses a microorganism such as Clostridium Ijungdahlii that is capable of consuming the carbon monoxide, carbon dioxide and hydrogen to produce ethanol and water. Finally, the ethanol is separated from the water in a distillation step. ClO₂ could be used as an antimicrobial agent in the fermentation step involving microorganisms such as Clostridium Ijungdahlii that are capable of consuming carbon monoxide, carbon dioxide and hydrogen to produce ethanol and water.

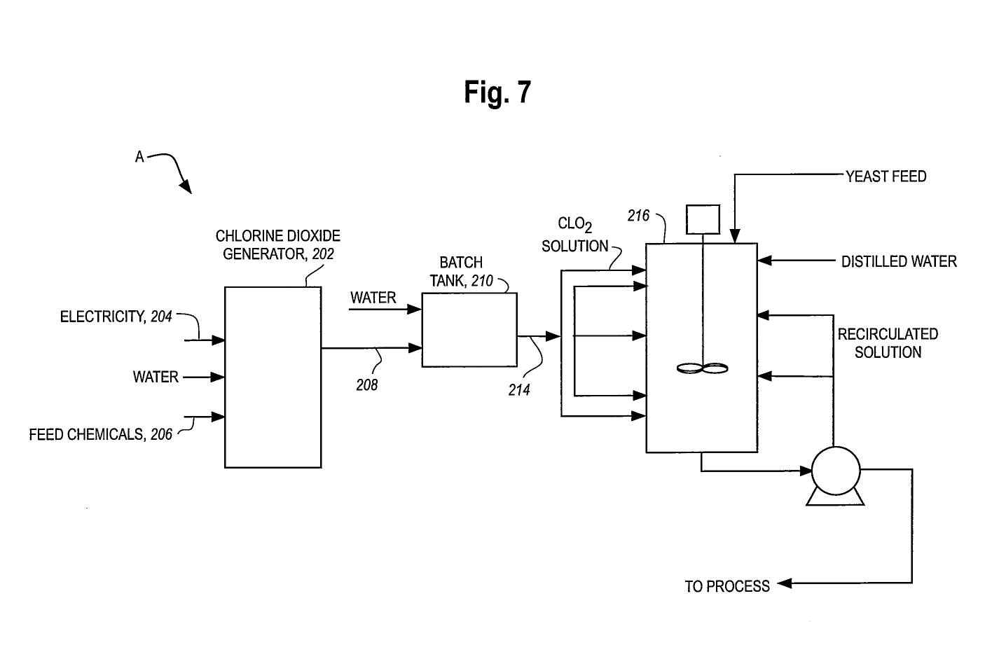

FIG. 7 illustrates an apparatus for carrying out the fermentation process with an integrated ClO₂ system.

The apparatus has a ClO₂ generator 202. The ClO₂ generator has an input for electricity 204. There is also an inlet for at least one chlorine containing chemical 206. There are three different types of chemical feed systems: a vacuum system, a pressure system and a combination system. Many types of feed systems can be employed to deliver chemicals in a fluid state. Chlorine gas, for example, can be added by a vacuum or combination feed system. The ClO₂ generator should also have an outlet for exhausting a ClO₂ gas stream 208 from the generator. In one embodiment the ClO₂ gas stream exiting the generator is between 0.0005 and 5.0% by weight in air.

For smaller scale production of fermentation products, skid-mounted equipment is ideal. Skid mounting allows the equipment to be manufactured off site, shipped to the desired location and easily installed. This ensures ease in transportation, faster erection and commissioning. The ClO₂ generator, batch tank, yeast vessel and connecting equipment could be made in a skid-mounted fashion.

A batch tank 210 that receives the ClO₂ gas stream is fluidly connected to the ClO₂ generator outlet 208. In the batch tank the ClO₂ gas is dissolved in water to form a ClO₂ solution. The batch tank has an inlet for introducing a water stream 212. The water stream and the ClO₂ gas stream are combined to form a ClO₂ solution. The concentration of the ClO₂ solution in the batch tank can vary across a wide range. Concentrations of up to about 5,000 mg/L can be achieved and concentrations of up to about 8,000 mg/L can be achieved with additional equipment. The ClO₂ solution is then exhausted from the batch tank through an outlet 214 at a specified dosage rate to create a solution of the desired concentration. In one embodiment the dosed ClO₂ solution, for application directly to the fermentation mixture, has a concentration of less than about 15 mg/L, preferably less than about 10 mg/L, and most preferable less than about 7.5 mg/L. In another embodiment the dosed ClO₂ solution, for application to the corn mash prior to fermentation, has a concentration of between about 10 and about 75 mg/L, preferably between about 10 and about 50 mg/L, and most preferable between about 20 and about 50 mg/L. In yet another embodiment the dosed ClO₂ solution, for use in propagation has a concentration of less than about 50 mg/L. In one embodiment, the exiting ClO₂ solution has an efficiency as ClO₂ in the stream of at least about 90%.

A yeast vessel 216 containing an aqueous yeast solution 218 is fluidly connected to the batch tank via the ClO₂ solution outlet 214. The yeast vessel could be a cook vessel, fermentation tank, conditioning tank, starter tank, propagation tank, liquefaction vessel and/or piping or heat exchanger between these units. Introducing the ClO₂ solution into the yeast vessel is capable of promoting propagation of yeast present while simultaneously decreasing the concentration of undesirable microorganisms.

While particular elements, embodiments and applications of the present invention have been shown and described, it will be understood, of course, that the invention is not limited thereto since modifications can be made by those skilled in the art without departing from the scope of the present disclosure, particularly in light of the foregoing teachings.

What is claimed is:

1. A method of reducing undesirable microorganism concentration, promoting yeast propagation/conditioning, and increasing yeast efficiency in an aqueous fluid stream employed in a fermentation process, the method comprising the steps of:
(a) introducing a quantity of fermentable carbohydrate to said stream;
(b) introducing a quantity of yeast to said stream;
(c) generating ClO₂ gas;
(d) dissolving said ClO₂ gas to form a ClO₂ solution;
(e) introducing an aqueous ClO₂ solution into said stream.

2. The method of claim 1 wherein said steps are performed sequentially.

3. The method of claim 1 wherein said ClO₂ gas is generated by reacting chlorine gas with water and then adding sodium chlorite.

4. The method of claim 1 wherein said ClO₂ gas is generated by reacting sodium hypochlorite with an acid and then adding sodium chlorite.

5. The method of claim 1 wherein said ClO₂ gas is generated by reacting sodium chlorite and hydrochloric acid.

6. The method of claim 1 wherein said ClO₂ gas is generated using an electrochemical cell and sodium chlorite.

7. The method of claim 1 wherein said ClO₂ gas is generated using an electrochemical cell and sodium chlorate.

8. The method of claim 1 wherein said ClO₂ gas is generated using an equipment-based sodium chlorate and hydrogen peroxide method.

9. The method of claim 1 wherein said ClO₂ solution has a concentration less than about 15 mg/L.

10. The method of claim 1 wherein said ClO₂ solution has a concentration between about 10 and about 75 mg/L.

11. The method of claim 1 wherein said ClO₂ solution has an efficiency as ClO₂ in the stream of at least 90%.

12. A method of reducing undesirable microorganism concentration, promoting yeast propagation/conditioning, and increasing yeast efficiency in an aqueous fluid stream employed in a fermentation process, the method comprising the steps of:
(a) introducing a quantity of fermentable carbohydrate to said stream;
(b) introducing a quantity of yeast to said stream; and
(c) introducing ClO₂ having an efficiency as ClO₂ of at least 90% into said stream.

13. The method of claim 12 wherein said steps are performed sequentially.

14. The method of claim 12 wherein said ClO₂ is an aqueous solution having a concentration less than about 15 mg/L.

15. The method of claim 12 wherein said ClO₂ is an aqueous solution having a concentration between about 10 and about 75 mg/L.

16. The method of claim 12 wherein said ClO₂ is a gas.

17. The method of claim 12 wherein said ClO₂ is produced by reacting sodium chlorate and hydrogen peroxide.

18. The method of claim 12 wherein said ClO₂ is produced by dry mix chlorine dioxide packets having a chlorite precursor packet and an acid activator packet.

19. The method of claim 12 wherein said ClO₂ is generated using an electrochemical cell and sodium chlorite.

20. The method of claim 12 wherein said ClO₂ is generated using an electrochemical cell and sodium chlorate.

21. The method of claim 12 wherein said ClO₂ is generated using an equipment-based sodium chlorate and hydrogen peroxide method.

22. An apparatus for reducing undesirable microorganism concentration, promoting yeast propagation/conditioning, and increasing fungi efficiency employed in a fermentation process, the apparatus comprising:
(a) a ClO₂ generator comprising an inlet for introducing at least one chlorine-containing feed chemical and an outlet for exhausting a ClO₂ gas stream from said generator;
(b) a batch tank fluidly connected to said ClO₂ generator outlet, said batch tank receiving said ClO₂ gas stream from said ClO₂ generator outlet, said batch tank comprising an inlet for introducing a second water stream and an outlet for exhausting an aqueous ClO₂ solution from said batch tank;
(c) a vessel for containing an aqueous fungi solution, said vessel fluidly connected to said batch tank;
wherein introducing said ClO₂ solution from said batch tank to said vessel promotes propagation of fungi present in said vessel.

23. The apparatus of claim 22 wherein said fungi vessel is heatable.

24. The apparatus of claim 22 wherein said fungi vessel is a fermentation tank having an inlet for fungi, an inlet for water, an inlet for fermentation chemicals and an outlet for the fermentation product connecting to processing equipment.

25. The apparatus of claim 22 wherein said fungi vessel is capable of performing liquefaction.

26. The apparatus of claim 22 wherein said fungi vessel is a yeast propagation tank.

27. The apparatus of claim 22 wherein said fungi vessel is a yeast conditioning tank.

28. The apparatus of claim 22 wherein said aqueous ClO₂ solution exhausted from said batch tank is dosed to a concentration less than about 15 mg/L.

29. The apparatus of claim 22 wherein said aqueous ClO₂ solution exhausted from said batch tank is dosed to a concentration between about 10 and about 75 mg/L.

30. The apparatus of claim 22 wherein said ClO₂ generator is skid mounted.

31. The apparatus of claim 22 wherein said batch tank is skid mounted.

32. The apparatus of claim 22 wherein said vessel for containing said aqueous fungi solution is skid mounted.

33. A method of reducing undesirable microorganism concentration, promoting desirable microorganism propagation/conditioning, and increasing desirable microorganism efficiency in an aqueous fluid stream employed in a fermentation process, the method comprising the steps of:
(a) introducing a quantity of cellulose to said stream;
(b) introducing a quantity of desirable microorganisms to said stream;
(c) generating ClO₂ gas;
(d) dissolving said ClO₂ gas to form a ClO₂ solution;
(e) introducing an aqueous ClO₂ solution into said stream.

34. The method of claim 33 wherein said steps are performed sequentially.

35. The method of claim 33 wherein said ClO₂ solution has an efficiency as ClO₂ in the stream of at least 90%.

36. A method of reducing undesirable microorganism concentration, promoting desirable microorganism propagation/conditioning, and increasing desirable microorganism efficiency in an aqueous fluid stream employed in a fermentation process, the method comprising the steps of:
(a) introducing a quantity of cellulose to said stream;
(b) introducing a quantity of desirable microorganisms to said stream; and
(c) introducing ClO₂ having an efficiency as ClO₂ of at least 90% into said stream.

37. The method of claim 36 wherein said steps are performed sequentially.

38. A method of reducing bacteria concentration without the use of antibiotics in an aqueous fluid stream employed in a fermentation process, the method comprising the steps of:
(a) introducing a quantity of desirable microorganisms to said stream; and
(b) introducing ClO₂ having an efficiency as ClO₂ of at least 90% into said stream.
